(19) United States
(12) Patent Application Publication
(10) Pub. No.: US 2021/0309677 A1
(43) Pub. Date: Oct. 7, 2021

(54) NOVEL AMINOPHOSPHINIC DERIVATIVES AS AMINOPEPTIDASE A INHIBITORS

(71) Applicants: QUANTUM GENOMICS, PARIS (FR); INSTITUT NATIONAL DE LA SANTE ET DE LA RECHERCHE MEDICALE, PARIS (FR); CENTRE NATIONAL DE LA RECHERCHE SCIENTIFIQUE, PARIS (FR); COLLEGE DE FRANCE, PARIS (FR)

(72) Inventors: FABRICE BALAVOINE, PARIS (FR); DELPHINE COMPERE, SCEAUX (FR); CATHERINE LLORENS-CORTES, BURES SUR YVETTE (FR)

(21) Appl. No.: 17/288,517

(22) PCT Filed: Oct. 25, 2019
(86) PCT No.: PCT/EP2019/079288

§ 371 Date: Apr. 24, 2021

(30) Foreign Application Priority Data
Oct. 26, 2018 (EP) 18306398.1

(51) Int. Cl. C07F 9/30; A61P 9/12

(52) U.S. Cl. CPC C07F 9/301; C07F 9/303; A61P 9/12

(57) ABSTRACT

The present invention relates to a novel compound, to a composition comprising the same, to methods for preparing the compound, and the use of this compound in therapy. In particular, the present invention relates to a compound that is useful in the treatment and prevention of primary and secondary arterial hypertension, ictus, myocardial ischaemia, cardiac and renal insufficiency, myocardial infarction, peripheral vascular disease, diabetic proteinuria, Syndrome X and glaucoma.

### FIELD OF THE INVENTION

The present invention relates to novel compounds, to a composition comprising the same, to methods for preparing the compounds, and the use of these compounds in therapy. In particular, the present invention relates to compounds that are useful in the treatment and prevention of primary and secondary arterial hypertension, ictus, myocardial ischemia, cardiac and renal insufficiency, myocardial infarction, peripheral vascular disease, diabetic proteinuria, Syndrome X and glaucoma.

### BACKGROUND OF THE INVENTION

Essential Hypertension (HTN) and Heart Failure (HF) are two of the major pathologies in cardio-vascular disease. HTN affects approximately 1 billion individuals worldwide. It is a leading risk factor for coronary heart disease, HF, stroke and renal insufficiency. Despite the availability of effective and safe drugs, HTN and its concomitant risk factors remain uncontrolled in many patients. HF remains the leading cause of hospitalization for patients over 65 years old in western countries. HF affects one to five persons in a thousand in industrialized countries, all ages considered, with a prevalence of three to twenty in a thousand. In the US, HF healthcare expenses represented $21 B in 2012, with the majority of costs related to hospitalizations. Despite the large number of drugs available HF has a poor prognosis as the one-year survival, all stages considered, is about 65%. HF remains one of the first causes of cardiovascular death, consequently, there is still an unmet medical need to develop new efficient and safe classes of drugs

The systemic renin-angiotensin system (RAS) is known to play a central role in blood pressure (BP) regulation and sodium metabolism. Systemic drugs targeting the RAS such as angiotensin I converting enzyme (ACE) inhibitors and angiotensin-II receptor type 1 (AT₁) antagonists are clinically effective in lowering BP and in preventing cardiovascular and renal morbidity and mortality in patients. Furthermore, activity of the renin-angiotensin aldosterone system (RAAS) is increased in patients with HF, and its maladaptive mechanisms may lead to adverse effects such as cardiac remodelling and sympathetic activation. Current evidence based guideline IA recommended medicines for HF with reduced ejection fraction are mainly RAAS-acting molecules such ACE inhibitors or AT₁ receptor blockers and beta-adrenergic receptor blocking agents.

A functional RAS controlling cardiovascular functions and body fluid homeostasis is also present in the brain. Several studies suggest that increased activity of the brain RAS results in an increase in sympathetic neuron activity and vasopressin release and that hyperactivity of the brain RAS plays a critical role in mediating high BP in various animal models of HTN as well as cardiac remodeling and dysfunction in animals models of HF (Marc Y, Llorens-Cortes, C Progress in Neurobiology 2011, 95, pp 89-103 ; Westcott K V et al, Can. J. Physiol. Pharmacol. 2009, 87, pp 979-988). Because recent evidences support that angiotensin III (Ang III) through its action on AT1 receptor may be the true peptide effector of the brain RAS for the central control of BP, the brain aminopeptidase A (APA) the enzyme generating Ang III from angiotensin II (Ang II) in the brain constitutes a promising therapeutic target for treatment of HTN and for the treatment of HF.

Aminopeptidase A (APA, EC 3.4.11.7) is a membrane-bound zinc metalloprotease, which has been characterized as the enzyme responsible for the conversion of AngII into AngIII in the brain (Zini S et al, Proc. Natl. Acad. Sci. USA 1996, 93, pp 11968-11973). Several APA inhibitors have been developed so far (Chauvel E N et al, J. Med. Chem. 1994, 37, pp 1339-1346; Chauvel E N et al, J. Med. Chem. 1994, 37, pp 2950-2957; David C et al, J. Med. Chem. 1999, 42, pp 5197-5211; Georgiadis D et al, Biochemistry 2000, 39, pp1152-1155; Inguimbert N et al, J. Peptide Res. 2005, 65, pp 175-188). Among them, EC33 ((3S)-3-amino-4-thiol-butyl sulfonate) was reported as a specific and selective APA inhibitor. Central infusions of EC33 were found to inhibit brain APA activity, to block the pressor responses to intracerebro-ventricular (icy) infusion of Ang II, and to lower BP in several experimental models of hypertension (Fournié-Zaluski M C et al Proc. Natl. Acad. Sci. USA 2004, 101, pp 7775-7780).

It was also further demonstrated that acute oral administrations in conscious hypertensive DOCA-salt rats and SHR rats of RB150 (also known as Firibastat) (15 to 150 mg/kg), a brain penetrating prodrug of EC33, induce a dose-dependent decrease in BP (Bodineau L et al, Hypertension 2008, 51, pp 1318-1325; Marc Y et al, Hypertension 2012, 60, pp 411-418). Interestingly, RB150 was found to lower BP in DOCA-salt rats and SHRs first by decreasing vasopressin release, increasing aqueous diuresis and natriuresis, thereby decreasing blood volume and BP to control values, and secondly by lowering sympathetic tone, thereby reducing vascular resistances and consequently decreasing BP. It was also reported that chronic central infusions of RB150, and the AT₁R blocker, losartan, are similarly effective in inhibiting sympathetic hyperactivity and cardiac dysfunction observed in rats with HF post MI (Huang B S et al, Cardiovascular Res. 2013, 97, pp 424-431). Thus, RB150 constitutes the first orally APA inhibitor able to enter the brain, block brain APA activity and normalize BP in hypertensive rats, and as such brain APA inhibitors represent a new class of centrally-acting agents for the treatment of HTN and HF.

The present inventors have now identified novel compounds which act as potent APA inhibitors and to that respect can be effective in reducing arterial hypertension and can have utility in treating arterial hypertension and the diseases to which it indirectly and directly contributes such as heart failure. Said compounds also present a satisfactory bioavailability and pharmacokinetics parameters, which makes them good candidates for oral or parenteral administration.

### SUMMARY OF THE INVENTION

Accordingly, the invention provides a compound with the following formula (I):

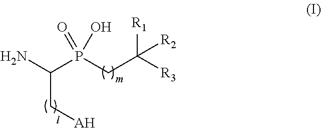

and more specifically having the following formula (II):

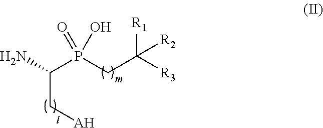

wherein:

AH represents —CO₂H, —SO₃H, —PO₃H₂;

I is 2 or 3;

m is 0, 1, 2 or 3;

R₁ represents a halogen atom, an alkyl group, a haloalkyl group, an alkoxy group, a haloalkoxy group, an O-cycloalkyl group, an O-aryl group, an O-arylalkyl group, a heteroalkyl group, an amino group optionally mono or disubstituted by an alkyl group, an haloalkyl group, a cycloalkyl group, an acyl group, an aryl group or an arylalkyl group;

R₂ and R₃ represent indepently a hydrogen atom, a halogen atom, an alkyl group, a haloalkyl group or can form together with the adjacent carbon atom depicted on figure (I) or (II) a cycloalkyl group;

a pharmaceutical salt, solvate, zwitterionic form or prodrug thereof.

In another aspect, the present invention discloses a composition comprising said compound of formula (I) and more specifically of formula (II). The composition is more particularly a pharmaceutical composition. The present invention provides therefore a pharmaceutical composition comprising at least one compound of the invention, preferably in association with a pharmaceutically acceptable diluent or carrier.

According to another aspect, the invention relates to a method for prevention or treatment of arterial hypertension and indirectly and directly related diseases, comprising administration of a therapeutically effective amount of a compound of this invention. In another aspect, the present invention provides a compound of the invention for use in therapy or medicine, and in particular, in human medicine, and more specifically for the treatment of arterial hypertension or indirectly and directly related diseases or disorders.

In another aspect, the present invention provides the use of a compound of the invention for the manufacture of a medicament for the treatment of arterial hypertension or indirectly and directly related diseases or disorders.

In another aspect, the present invention provides a method of treatment of a patient suffering from arterial hypertension or indirectly and directly related diseases comprising the administration of a therapeutically effective amount of a compound of the invention in a patient in need thereof.

### DETAILED DESCRIPTION

The present invention thus relates to a compound having the following formula (I):

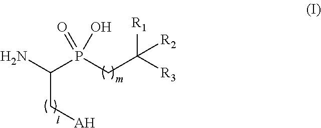

and more specifically having the following formula (II):

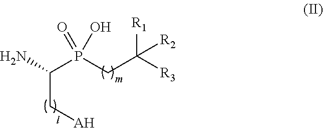

wherein:

AH represents —CO₂H, —SO₃H, —PO₃H₂;

I is 2 or 3;

m is 0,1, 2 or 3;

R₁ represents a halogen atom, an alkyl group, a haloalkyl group, an alkoxy group, a haloalkoxy group, an O-cycloalkyl group, an O-aryl group, an O-arylalkyl group, a heteroalkyl group, an amino group optionally mono or disubstituted by an alkyl group, an haloalkyl group, a cycloalkyl group, an acyl group, an awl group or an arylalkyl group;

R₂ and R₃ represent independently a hydrogen atom, a halogen atom, an alkyl group, a haloalkyl group or can form together with the adjacent carbon atom depicted on formula (I) or (II) a cycloalkyl group.

The present invention provides methods of prevention or treatment of arterial hypertension and diseases to which arterial hypertension directly or indirectly contributes. Such diseases include diseases of the heart, the peripheral and cerebral vascular system, the brain, the eye and the kidney. In particular diseases include primary and secondary arterial hypertension, ictus, myocardial ischemia, cardiac and renal insufficiency, myocardial infarction, peripheral vascular disease, diabetic proteinuria, Syndrome X and glaucoma.

As used herein, “a compound of the invention” means a compound described above or a prodrug thereof or a pharmaceutically acceptable salt, solvate or any zwitterionic form thereof.

Within the context of the present invention:

The term “alkyl” or “Alk” means a monovalent or divalent, linear or branched, saturated hydrocarbon chain, having from 1 to 8 carbon atoms (also named (C₁-C₈)alkyl), such as methyl, ethyl, propyl, isopropyl, n-butyl, iso-butyl, sec-butyl, tert-butyl, tert-butyl-methyl, n-pentyl, n-hexyl, n-heptyl, or n-octyl group.

The term “acyl” means a —C(O)R group where R is an alkyl group as defined earlier or a phenyl group. Acyl group includes for example acetyl, ethylcarbonyl, or benzoyl group.

The term “alkoxy” or “alkyloxy” means a —OAlk group wherein Alk is an alkyl group as defined earlier. Alkoxy group includes for example methoxy, ethoxy, n-propyloxy, or tert-butyloxy group.

The term “aryl” means an aromatic monocyclic or bicyclic system having from 4 to 10 carbon atoms (also named (C₄-C₁₀)aryl), it being understood that in the case of a bicyclic system, one of the cycles is aromatic and the other cycle is aromatic or unsaturated. Aryl groups include for example phenyl, naphthyl, indenyl, or benzocyclobutenyl groups.

The term “arylalkyl” means a -Alk-Ar group (i.e. an aryl group connected to the remainder of the molecule by an alkyl group), wherein Alk represents an alkyl group as defined above, and Ar represents an aryl group as defined above.

The term “heteroalkyl” means a linear or branched saturated hydrocarbon chain, having from 1 to 5 carbon atoms and at least 1 or 2 heteroatoms, such as sulfur, nitrogen or oxygen atoms. Heteroalkyl for example includes —O(CH₂)₂OCH₃ or —(CH₂)₂OCH₃ group.

The term “halogen atom” means fluorine, bromine, chlorine or iodine atom.

The term “cycloalkyl” means a saturated monocyclic or polycyclic system, such as a fused or bridged bicyclic system, having from 3 to 12 carbon atoms (also named (C₃-C₁₂)cycloalkyl), such as cyclopropyl, cyclobutyl, cyclopentyl, cyclohexyl, cycloheptyl, cyclooctyl, adamantly, decalinyl, or norbornyl groups.

The term “O-cycloalkyl” means a cycloalkyl group as defined earlier connected to the remainder of the molecule through an oxygen atom. O-cycloalkyl includes for example the O-cyclopentyl or O-cyclohexyl group.

The term “O-aryl” means an aryl group as defined earlier connected to the remainder of the molecule through an oxygen atom. O-aryl comprises for example the O-phenyl group.

The term “O-arylalkyl” means an arylalkyl group as defined earlier connected to the remainder of the molecule through an oxygen atom. O-arylalkyl includes for example the O-benzyl group.

The “ester” means a —C(O)OR group with R representing an alkyl, aryl or arylalkyl group as defined earlier.

The term “haloalkyl” means a linear or branched saturated hydrocarbon chain, having from 1 to 6 carbon atoms and substituted with one or more, and notably 1-6 halogen atoms, such as the trifluoromethyl or 2,2,2-trifluoroethyl groups.

The term “haloalkoxy” means a linear or branched saturated hydrocarbon chain, having from 1 to 6 carbon atoms and substituted with one or more, and notably 1-6 halogen atoms, said chain being connected to the compound through an oxygen atom, such as the trifluoromethoxy or 2,2,2-trifluoroethoxy groups.

The term “amino group” means a —NH₂ group optionally mono or disubstituted by an alkyl group, as defined above.

The term “protective group” or “protection group” means the group which selectively blocks the reactive site in a multifunctional compound so that a chemical reaction may be selectively carried out at another non-protected reactive site, with the meaning conventionally associated with the latter in synthesis chemistry.

In the present invention, the term “pharmaceutically acceptable” refers to which can be used in the preparation of a pharmaceutical composition which is generally safe, non-toxic and not undesirable, biologically or otherwise, and which is commonly accepted for a veterinary or human pharmaceutical use.

The term “pharmaceutically acceptable salts” of the compounds of the invention include conventional salts formed from pharmaceutically acceptable inorganic or organic acids or bases as well as quaternary ammonium salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic etc. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. For example, preferred salt forms include chlorhydrate.

The term “pro-drug” means a chemical derivative of the compound, object of the invention, which generates in vivo said compound by a spontaneous chemical reaction with the physiological medium, notably by an enzymatic reaction, a photolysis and/or a metabolic reaction. In the present case, pro-drug of the compounds of the invention generate in vivo compounds identified as inhibitors of aminopeptidase A.

A pro-drug may be obtained by derivatizing functional group with specific labile moieties. The pro-drug with an acid function (such as phosphinic acid, carboxylic acid, sulfonic acid or phosphonic acid) notably comprises ester, the pro-drug with amine function notably comprises [(2-methylpropanoyl)oxy]ethoxycarbonyl via a carbamate group or comprises 2-oxo-[1,3-thiazolidine-4-yl]formamide via an amide group.

Other examples are described in T. Higuchi and V. Stella, “Pro-drugs as Novel Delivery system”, Vol.14, A.C.S Symposium Series, American Chemical Society (1975) and “Bioreversible Carriers in Grug Design: Theroy and Application”, edited by E. B. Roche, Pergamon Press: New York, 14-21 (1987).

The term “isomer” refers to compounds which have identical molecular formulae as identified herein but which differ by nature or in the binding sequence of their atoms or in the layout of their atoms in space. Isomers which differ in the layout of their atoms in space are designated by “stereoisomers”. Stereosiomers which are not mirror images of each other, are designated as “diastereoisomers”, and stereoisomers which are non-superposable mirror images of each other are designated as “enantiomers” or “optical isomers”. “Stereoisomers” refer to racemates, enantiomers and diastereoisomers.

The person skilled in the art will recognize that stereocenters exist in the compounds of the invention. Any chiral center of a compound of the invention can be (R), (S) or racemate. Accordingly, the present invention includes all possible stereoisomers and geometric isomers of the compounds of formula (I) and includes not only racemic compounds but also the optically active isomers as well. According to a preferred embodiment, compounds of the invention is of formula (II). When a compound of formula (I) is desired as a single enantiomer, it may be obtained either by resolution of the final product or by stereospecific synthesis from either isomerically pure starting material or any suitable intermediate. Resolution of the final product, an intermediate or a starting material may be effected by any suitable method known in the art. See, for example, Stereochemistry of Carbon Compounds by E. L. Eliel (Mcgraw Hill, 1962) and Tables of Resolving Agents by S. H. Wilen.

The person skilled in the art will recognize that the compounds of the invention may contain at least one positive and one negative charge so that the compounds of the invention includes zwitterionic forms thereof. In chemistry, a zwitterion (also called an inner salt), is a molecule with two or more functional groups, of which at least one has a positive and one has a negative electrical charge and the charges on the different functional groups balance each other out, and the molecule as a whole is electrically neutral. The pH where this happens is known as the isoelectric point. Accordingly, any zwitterionic forms of the compounds of the invention including prodrugs thereof are within the scope of the present invention.

The specialist in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of formula (I) or (II) are within the scope of the present invention.

It will also be appreciated by the specialist in organic chemistry that many organic compounds can exist in more than one crystalline form. For example, crystalline form may vary from solvate to solvate. Thus, all crystalline forms of the compounds of the invention or the pharmaceutically acceptable solvates thereof are within the scope of the present invention.

References herein to a compound according to the invention include both compounds of formula (I) or (II) and their pharmaceutically acceptable salts, solvates, or prodrugs.

According to preferred embodiments, the compounds of the present invention correspond to general formula (I) and more specifically formula (II), wherein:

m is 0 or 1; and/or

AH is CO₂H or SO₃H or PO₃H₂; and/or

R₁ represents a halogen atom, an alkyl group, a haloalkyl group, an alkoxy group, a haloalkoxy group, an O-cycloalkyl group, an O-aryl group, an O-arylalkyl group, a heteroalkyl group, an haloalkyl group, a cycloalkyl group, an acyl group, an aryl group or an arylalkyl group.

References herein to a compound according to the invention include both compounds of formula (I) or (II) and their pharmaceutically acceptable salts, solvates, zwitterionic forms or pro-drugs.

According to a particular embodiment, a pro-drug of the compound according to the invention can be a product having the following formula (III):

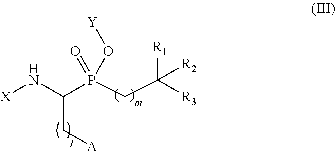

and more specifically, the following formula (IV):

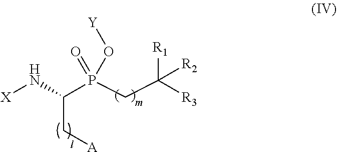

wherein:

I, m, R₁, R₂, R₃ are as defined above;

A represents —SO₃Z —CO₂Z or —P(O)(OZ)₂, with Z selected from the group consisting of a hydrogen atom, an alkyl and arylalkyl group;

X represents a hydrogen atom, —(CO)-alkyl, —(CO)-alkoxy, —(CO)-benzyloxy,

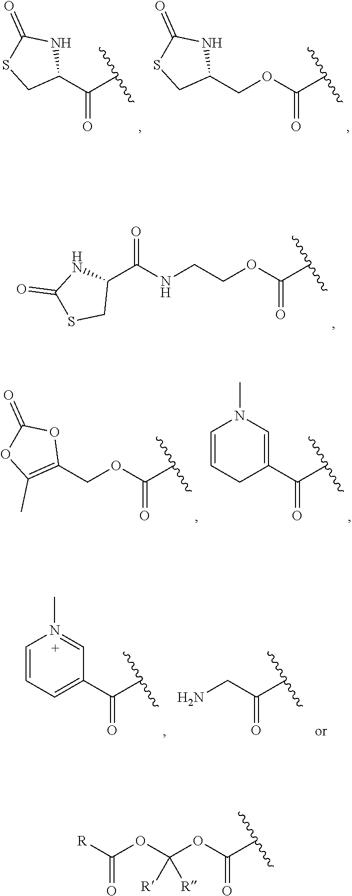

with R represents an alkyl group and, R′ and R″ represent indepently a hydrogen atom or an alkyl group;

Y represents a hydrogen atom, an alkyl, aryl, arylalkyl or

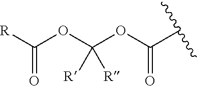

with R, R′ and R″ as defined above,

wherein at least one of Z, X and Y is different from hydrogen atom.

According to specific embodiments, the compound of the invention is selected from the group consisting of: 4-amino-4-[hydroxy(3-methylbutyl)phosphoryl]butanoic acid, 4-amino-4-[hydroxy(4-methylpentyl)phosphoryl]butanoic acid, 4-amino-4-[(2-cyclohexylethyl)(hydroxy)phosphoryl]butanoic acid, 4-amino-4-[hydroxy(pentyl)phosphoryl]butanoic acid, 4-amino-4-[hexyl(hydroxy)phosphoryl]butanoic acid, 4-amino-4-[(cyclobutylmethyl)(hydroxy)phosphoryl]butanoic acid, 4-amino-4-[(cyclopentylmethyl)(hydroxy)phosphoryl]butanoic acid, 4-amino-4-[hydroxy(5-methylhexyl)phosphoryl]butanoic acid, 4-amino-4-[hydroxy(4,4,4-trifluorobutyl)phosphoryl]butanoic acid, 4-amino-4-[(cyclohexylmethyl)(hydroxy)phosphoryl]butanoic acid, and 4-amino-4-[hydroxy({[(propan-2-yl)amino]methyl})phosphoryl]butanoic acid.

The compounds of the invention are conveniently administered in the form of pharmaceutical compositions. Such compositions may conveniently be presented for use in conventional manner in admixture with one or more physiologically acceptable carriers or excipients. The carrier(s) must be “acceptable” in the sense of being compatible with the other ingredients of the formulation and not deleterious to the subject receiving them.

While it is feasible that compounds of the present invention may be therapeutically administered as a raw chemical, it is also possible to present the active ingredient as a pharmaceutical formulation.

Accordingly, the present invention further provides for a pharmaceutical composition comprising a compound of the present invention in association with one or more pharmaceutically acceptable carriers and, optionally, other active ingredients.

The pharmaceutical compositions include those suitable for oral, parenteral (including subcutaneous e.g. by injection or by depot tablet, intradermal, intrathecal, intraocular, intramuscular e.g. by depot and intravenous), rectal and topical (including dermal (i.e. on the skin), buccal and sublingual) or in a form suitable for administration by inhalation or insufflation, although the most suitable route may depend upon for example the condition and disorder of the recipient. The compositions may conveniently be presented in unit dosage form and may be prepared by any of the methods well known in the art of pharmacy. All methods include the step of associating the compounds of the invention, optionally with at least one other active ingredient, with the carrier which constitutes one or more accessory ingredients. In general the formulations are prepared by uniformly and intimately associating the active ingredient with liquid carriers or finely divided solid carriers or both and then, if necessary, shaping the product into the desired formulation.

Pharmaceutical compositions suitable for oral administration may be presented as discrete units such as capsules, cachets or tablets (e.g. chewable tablets in particular for pediatric administration) each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a other conventional excipients such as binding agents, (for example, syrup, gum arabic, gelatin, sorbitol, tragacanth, mucilage of starch, polyvinylpyrrolidone or hydroxymethyl cellulose), fillers (for example, lactose, sucrose, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycolate) or wetting agents, such as sodium lauryl sulfate. Molded tablets may be made by molding in a suitable machine a mixture of the powdered compound moistened with an inert liquid diluent. The tablets may optionally be coated or scored and may be formulated so as to provide slow or controlled release of the active ingredient therein. The tablets may be coated according to methods well-known in the art.

Alternatively, the compounds of the present invention may be incorporated into oral liquid preparations such as aqueous or oily suspensions, solutions, emulsions, and such as syrups or elixirs, for example. Moreover, pharmaceutical compositions (or formulations) containing these compounds may be presented as a dry product for constitution with water or other suitable vehicle before use. Such liquid preparations may contain conventional additives such as suspending agents such as sorbitol syrup, methyl cellulose, glucose/sugar syrup, gelatin, hydroxyethylcellulose, carboxymethyl cellulose, aluminum stearate gel or hydrogenated edible fats; emulsifying agents such as lecithin, sorbitan mono-oleate or arabic gum; non-aqueous vehicles (which may include edible oils) such as almond oil, fractionated coconut oil, oily esters, propylene glycol or ethyl alcohol; and preservatives such as methyl or propyl p-hydroxybenzoates or sorbic acid. These preparations may also be formulated as suppositories, e.g., containing conventional suppository excipients such as cocoa butter or other glycerides.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of a sterile liquid carrier, for example, water-for-injection, immediately prior to use. Extemporaneous injection solutions and suspensions may be prepared from sterile powders, granules and tablets of the kind previously described.

Compositions for rectal administration may be presented as a suppository with the usual carriers such as cocoa butter, hard fat or polyethylene glycol.

Formulations for topical administration in the mouth, for example buccally or sublingually, include lozenges comprising the active ingredient in a flavored excipient such as sucrose and arabic gum or tragacanth, and pastilles comprising the active ingredient in an excipient such as gelatin and glycerin or sucrose and arabic gum. For topical administration onto the skin, the compounds may be formulated as creams, gels, ointments or lotions or as a transdermal patch. For ocular administration, the compositions can be a liquid solution (such as eye-drop solution), a gel, a cream or any type of ophthalmic compositions.

The compounds may also be formulated as depot preparations. These long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For intranasal administration the compounds of the present invention may be used, for example as a liquid spray, as a powder or in the form of drops.

For administration by inhalation the compounds according to the present invention are conveniently delivered in the form of an aerosol spray presentation from a pressurised container or a nebuliser, with the use of a suitable propellant, e.g. 1,1,1,2-trifluoroethane (HFA 134A) and 1,1,1,2,3,3,3, -heptafluoropropane (HFA 227), carbon dioxide or other suitable gas. In the case of a pressurised aerosol the exact dosage may be determined by providing a valve adapted to deliver a metered amount. Capsules and cartridges of e.g. gelatin for use in an inhaler or insufflator may be formulated so as to contain a powder mix of a compound of the present invention and a suitable powder excipient such as lactose or starch.

In addition to the ingredients particularly mentioned above, the formulations may include other agents conventional in the art having regard to the type of formulation in question, for example those suitable for oral administration may include flavoring agents.

It will be appreciated by the person skilled in the art that reference herein to treatment extends to prophylaxis as well as the treatment of established diseases or symptoms. Moreover, it will be appreciated that the amount of a compound of the present invention required for use in treatment will vary with the nature of the condition being treated and the age and the condition of the patient and will be ultimately at the discretion of the attendant physician or veterinarian. In general, however, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day. The formulations according to the present invention may contain between 0.1-99% of the active ingredient, conveniently from 30-95% for tablets and capsules and 3-50% for liquid preparations.

The compounds of the present invention for use in the present invention may be used in association with one or more other therapeutic active agents for example, beta-adrenergic receptor antagonists, calcium channel blocking agents, thiazide diuretics, angiotensin receptor antagonists and angiotensin converting enzyme inhibitors. The present invention thus provides in a further aspect the use of a combination comprising a compound of the invention with a further therapeutic agent in the treatment of arterial hypertension.

When the compounds of the present invention are used in association with other therapeutic agents, the compounds may be administered either sequentially or simultaneously by any suitable route.

The associations referred to above may suitably be presented for use in the form of a pharmaceutical formulation and thus pharmaceutical formulations comprising an association as defined above optimally together with a pharmaceutically acceptable carrier or excipient are a further aspect of the present invention. The individual components of such associations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any suitable formulation, suitably in a manner known for such compounds in the art.

When a compound of the present invention is used in association with a second therapeutic agent active against the same disease, the dose of each compound may differ from that administered when the compound is used alone. Appropriate doses will be readily determined by the person skilled in the art.

In another aspect, a subject of the present invention is a method for the prevention or treatment of arterial hypertension and of directly and indirectly related diseases, comprising the administration of a therapeutically effective amount of a compound of the present invention.

In another aspect, the present invention provides compounds of the present invention for use in therapeutics, and in particular in veterinary or human medicine.

The invention also relates to the use of a compound of formula (I) or (II), as a selective inhibitor with regard to aminopeptidase A.

In another aspect, the present invention provides the use of a compound of the present invention, for producing a medicinal product for use in the treatment of arterial hypertension and of directly and indirectly related diseases.

In another aspect, the present invention provides a method of treating a patient suffering from arterial hypertension and from directly and indirectly related diseases, comprising the administration of a therapeutically effective amount of a compound of the present invention.

The present invention provides methods for the prevention or treatment of arterial hypertension and of diseases to which arterial hypertension directly or indirectly contributes. These diseases comprise heart disease, heart failure, stroke, peripheral and/or cerebral vascular system diseases, and brain, eye and kidney diseases. In particular, the diseases comprise primary and secondary arterial hypertension, an ictus, myocardial ischemia, cardiac insufficiency and renal insufficiency, myocardial infarction, a peripheral vascular disease, diabetic protinuria, syndrome X, glaucoma, neurodegenerative diseases and memory disorders.

The compounds of formula (I) or preferably (II) can be prepared by several methods. The starting products are commercial products or products prepared according to known synthesis from commercial compounds or known to one skilled in the art. More specifically, the method for preparing the compound of the invention comprises the following successive steps:

The compounds of formula (I), objects of the present invention, may be prepared according to the synthesis route described hereafter, by using precursors of the following formulae (V), (VI) and (VII),

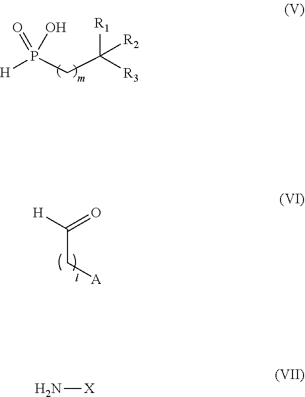

wherein I, m, R₁, R₂, R₃, A and X are defined above. According to this synthesis route, a multi-component reaction is carried out between the compounds (V), (VI) and (VII) for example in the presence of acetic acid and acetyl chloride in organic solvent such as toluene in order to lead to the compound of formula (VIII):

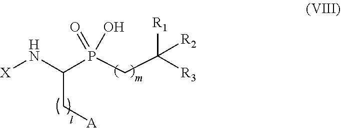

Next, simultaneously deprotection of the protecting group of function A and the protecting group X of amino function could occur by hydrogenolysis to lead to the formation of the compound of the present invention of formula (I).

In some cases, the group A of the compound of formula (VIII) is selectively de-protected by lithine for example to provide the intermediate compound of formula (IX),

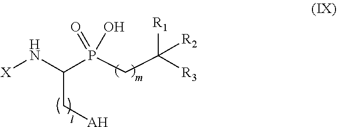

Next, the compound of formula (IX) is submitted to hydrogenolysis or to acidic conditions such as trifluoroacetic acid in organic solvent like anisole under heating to provide the compound of the present invention of formula (I).

The compounds of formula (I), objects of the present invention, may also be prepared according to the synthesis route described hereafter, by using precursors of the following formulae (Vbis) and (X),

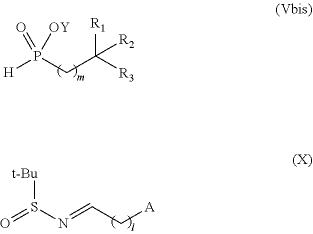

wherein I, m, Y, R₁, R₂, R₃ and A are defined above. According to this synthesis route, a reaction is carried out between the compound (Vbis) and the sulfo-imine (X), obtained by well-known methods from the literature, in the presence for example of cesium carbonate in organic solvent such as dichloromethane in order to lead to the compound of formula (XI):

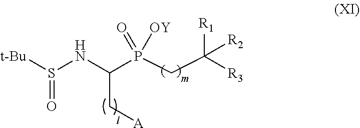

wherein I, m, Y, R₁, R₂, R₃ and A are as defined above.

It is worthy to note that sulfo-imine intermediate (X) could be synthesized in chiral form by well-known methods of the literature. When chiral inductor protecting group is supported by sulfo-imine (X), this synthon could provide access to asymmetric synthesis of precursor of compound of formula (II).

Appropriate deprotection steps applied to intermediate (XI) in racemate form or chiral form provide access to compounds of the invention of formula (I) or (II) respectively.

The precursor of formula (V) may be obtained from the compound of the following formula (XII),

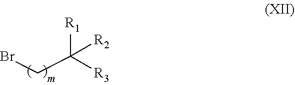

by reacting the corresponding Grignard reagent with diethylchlorophosphite in organic solvent like diethyl ether or tetrahydrofuran in cooled conditions such as 0-10° C.

The following examples illustrate the invention but do not limit it by any means.

### EXAMPLES

The starting products used are commercial products or products prepared according to known synthesis from commercial compounds or known to one skilled in the art. The different general procedures A, B, C, lead to synthesis intermediates useful for preparing the compounds of the invention. Procedures D and E lead to synthesis of final compounds of the invention. The structures of the compounds described in the examples were determined according to the usual spectrophotometric techniques (nuclear magnetic resonance (NMR), mass spectrometry including electrospray ionisation (ESI⁻) . . . ) and purity was determined by high performance liquid chromatography (HPLC).

Synthesis intermediates and compounds of the invention are named according to the IUPAC (The International Union of Pure and Applied Chemistry) nomenclature and described in their neutral form.

The following abbreviations have been used:

AIBN: azobisisobutyronitrile

(Boc)₂O: di-tert-butyl dicarbonate

(n-Bu)₄NBr: tetra-n-butylammonium bromide

(n-Bu)₄NI: tetra-n-butylammonium iodide

AcCl: acetyl chloride

AcOH: acetic acid

BTSP: bis(trimethylsilyl)phosphonate

Cbz: carboxybenzyl

CH₂Cl₂ or DCM: dichloromethane

CHCl₃: chloroform

cHex: cyclohexane

CuSO₄: copper sulfate

DCC: N,N′-dicyclohexylcarbodiimide

DTAD : di-tert-butyl azodicarboxylate

EDCI: 1-ethyl-3-(3-dimethylaminopropyl)ethylcarbodiimide

Et₂O: diethyl ether

EtOAc: ethyl acetate

HBF₄.Et₂O: tetrafluoroboric acid diethyl ether complex

HCl: hydrochloric acid

HMDS: 1,1,1,3,3,3-Hexamethyldisilazane

I₂: iodine

i-PrOH: isopropanol

K₂CO₃: potassium carbonate

KOtBu: potassium tert-botuxide

LiAlH₄: lithium aluminium hydride

LiHMDS: lithium bis(trimethylsilyl)amide

LiOH.H₂O: lithium hydroxide monohydrate (lithine)

MeOH: methanol

Mg: magnesium

Na₂S₂O₃: sodium thiosulfate

Na₂SO₄: sodium sulfate

NaBH₄: sodium borohydride

NaHCO₃: sodium bicarbonate

NEt₃: tritethylamine

NH₂Cbz: benzyl carbamate

NH₄Cl: ammonium chloride

Pd(PPh₃)₄: Tetrakis(triphenylphosphine)palladium(0)

TFA: trifluoroacetic acid

Eq.: equivalent

ESI: Electrospray Ionisation

HPLC: High Performance Liquid Chromatography

NMR: Nuclear Magnetic Resonance

PTFE filter: polytetrafluoroethylene filter

### General Procedure for the Preparation of Intermediate (V) (Procedure A)

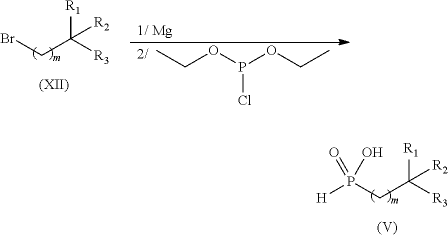

Intermediate (XII), transformed to the corresponding Grignard solution (0.5 to 1.0 M in anhydrous THF or Et₂O, 1.05 eq.), was added dropwise to a cooled solution (5° C.) of diethylchlorophosphite (1.0 eq.) in anhydrous Et₂O (1.3 mL/mmol of diethylchlorophosphite), under argon atmosphere, maintaining the internal temperature between 0-10° C. during the addition. After 16 h of stirring at room temperature, the mixture was filtered through celite. The filtrate was concentrated under reduced pressure. The residue was dissolved in water and treated with concentrated aqueous HCl (pH=1). The resulting mixture was stirred at room temperature until a colorless transparent solution was obtained (15 min). This solution was extracted with EtOAc (three times) and the combined organic layers were washed with brine, dried over Na₂SO₄, filtered and concentrated in vacuo. The clear liquid was diluted in aqueous NaOH 2 M and the resulting solution was stirred for 1 h. The aqueous layer was washed with Et₂O, and then acidified with concentrated HCl (until pH=1). The resulting acid aqueous layer was extracted with DCM (three times). The combined organic layer was dried over Na₂SO₄, filtered, and concentrated under vacuum to afford the desired intermediate (V).

### General Procedure for Multi-Component Reaction (Procedure B)

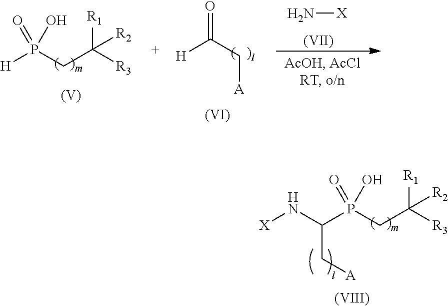

To a solution of intermediate (V) (1.0 eq.) and benzyl carbamate (VII) (H₂N—X with X═CBz) (1.1 eq.) in a mixture ^{˜}6:1 of AcOH (0.9-1.8 mL/mmol of intermediate (V)) and AcCl (0.09-0.52 mL/mmol of intermediate (V)) was added dropwise intermediate (VI) (1.2 eq.). After 18 h of stirring at room temperature, the reaction mixture was co-evaporated with toluene (three times). The residue was taken up in DCM, water was then added to quench the remaining AcCl and then the aqueous layer was extracted with DCM (three times). The combined organic layers were dried over Na₂SO₄, filtered and concentrated in vacuo. The crude material was triturated in Et₂O, filtered and the obtained solid was dried to afford the desired intermediate (VIII).

### General Procedure for Selective Deprotection (Procedure C)

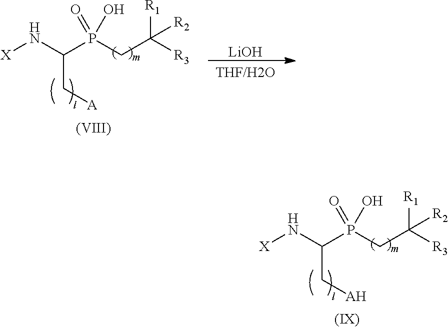

To intermediates (VIII) (1.0 eq.) in a mixture of THF/water (4:1) was added LiOH.H₂O (3.0 eq.) in one portion. The mixture took instantaneously an orange coloration and was stirred at room temperature until completion of reaction. The mixture was concentrated to evaporate THF, then the aqueous layer was extracted with EtOAc (three times). The aqueous layer was then acidified to pH 1 with HCl aqueous solution while a precipitate appeared. Most of the time, the aqueous layer was extracted with DCM (five times) and the combined organic layers were dried over Na₂SO₄, filtered and concentrated in vacuo to afford the corresponding selectively deprotected intermediate (IX). In some cases, the precipitate obtained after acidic treatment was directly filtered and dried to afford the expected intermediate.

### General Procedure for Final Deprotection in Acidic Conditions (Procedure D)

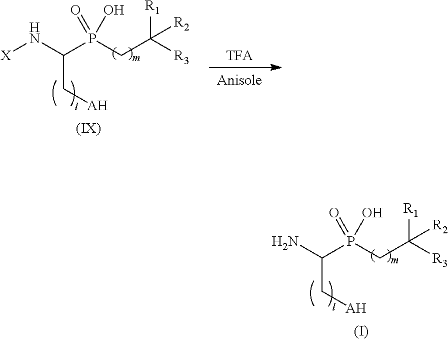

To intermediate (IX) selectively deprotected according to procedure C were added TFA/anisole. The resulting solution was stirred at 75° C. for 2 to 6 h with TFA/anisole conditions then at room temperature if needed. After concentration and co-evaporation with toluene (three times), or direct filtration in the case where a precipitate appears, the crude was purified by trituration, preparative LCMS or reverse phase column to afford the desired compound of the invention of formula (I).

### General Procedure for Hydrogenolysis (Procedure E)

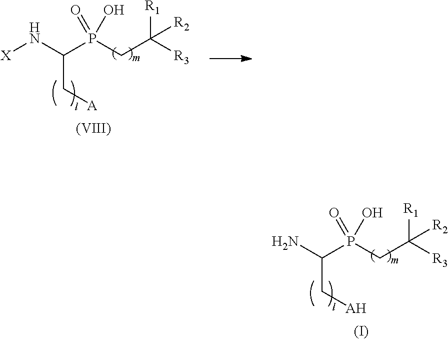

The intermediate (VIII) (1.0 eq.) was dissolved in a mixture of EtOH/AcOH or MeOH/AcOH (global volume: 17-34 mL/mmol of protected compound, depending on its solubility). The powder was sonicated to promote solubility and the clear solution was then submitted to H-Cube (catalyst=10% Pd/C, T=40° C., flow rate=0.6-0.8 mL/min, full H₂ mode or 10 bars). After concentration, the crude was purified by trituration or by reverse phase column to afford the desired compound of the invention of formula (I).

### Preparation of benzyl 4-oxobutanoate

### Step 1: Synthesis of benzyl 4-hydroxybutanoate

Gamma-butyrolactone (20 mL, 255 mmol, 1.0 eq.) and NaOH (10.2 g, 255 mmol, 1.0 eq.) were dissolved in water (170 mL) and the temperature was raised to 70° C. After 12 hours, water was evaporated and the white paste was included with the toluene evaporated (three times). The white solid was placed under vacuum and heated to 70° C. for 2 hours. The solid was taken up again with toluene to remove any trace of water. The obtained white solid was suspended in acetone (280 mL). Tetrabutylammonium iodide (4.72 g, 12.8 mmol, 0.05 eq.) and benzyl chloride (29.4 mL, 255 mmol, 1.0 eq.) were added to the suspension. The solution was refluxed for 6 h and then go back at room temperature overnight. The reactional mixture was then refluxed again during 6 h. At room temperature, the mixture was filtered and the filtrate was evaporated to give the crude which was purified by chromatography on silica gel. The fractions containing expected product were combined and concentrated in vacuo to afford the title product (36.5 g, 74%).

¹H NMR (CDCl₃, 500 MHz) δ (ppm): 7.39-7.31 (m, 5H); 5.13 (s, 2H); 3.69 (t, 2H, J=6.0 Hz); 2.50 (t, 2H, J=7.0 Hz); 1.93-1.88 (m, 2H)

### Step 2: Synthesis of benzyl 4-oxobutanoate

Benzyl 4-hydroxybutanoate (10 g, 51.49 mmol, 1.0 eq.) was dissolved in dichloromethane (1.7 L) and cooled to 0° C. Dess-Martin periodinane (33 g, 77.23 mmol, 1.5 eq.) was added and the mixture was stirred at room temperature for 2 h30. The mixture was concentrated and the crude was purified by flash chromatography on silica gel. The fractions containing expected product were combined and concentrated in vacuo to afford the title compound (8.0 g, 81%) as a light yellow oil.

¹H NMR (CDCl₃, 500 MHz) δ (ppm): 9.82 (s, 1H); 7.39-7.31 (m, 5H); 5.14 (s, 2H); 2.82 (t, 2H, J=7.0 Hz); 2.71-2.67 (m, 2H)

### Example 1

### 4-amino-4-[hydroxy(3-methylbutyl)phosphoryl]butanoic acid

### Step 1: (3-methylbutyl)phosphinic acid

The title compound (1.40 g, 59%) was prepared according to the procedure A from diethylchlorophosphite (1.90 mL, 17.4 mmol, 1.0 eq.) in anhydrous Et₂O (6 mL) followed by addition of the freshly prepared Grignard reagent from 1-bromo-3-methylbutane (2.76 g, 18.3 mmol, 1.05 eq.) in anhydrous Et₂O (9 mL).

MS (ESI⁺): [M+H]⁺=137.2; [(M×2)+H]⁺=273.2

¹H NMR (MeOD, 500 MHz) δ (ppm): 7.02 (dt, J=536.2, 2.0 Hz, 1H); 1.85-1.71 (m, 2H); 1.71-1.59 (m, 1H); 1.55-1.42 (m, 2H); 0.96 (d, J=6.7 Hz, 6H)

³¹P NMR (CD₃OD, 202 MHz) δ (ppm): 36.32

### Step 2: [4-(benzyloxy)-1-{[(benzyloxy)carbonyl]amino}-4-oxobutyl](3-methylbutyl) phosphinic acid

The title compound (1.75 g, 65%) obtained as a white solid was prepared according to the procedure B for multi-component reaction from previous product (800 mg, 5.88 mmol, 1.0 eq.) and NH₂Cbz (977 mg, 6.46 mmol, 1.1 eq.) in AcOH (10 mL) and AcCl (1.2 mL) followed by addition of benzyl 4-oxobutanoate (1.36 g, 7.05 mmol, 1.2 eq.).

MS (ESI⁺): [M+H]⁺=462.2; [(M×2)+H]⁺=923.6

¹H NMR (CD₃OD, 500 MHz) δ (ppm): 7.54-7.22 (m, 10H); 5.23-5.02 (m, 4H); 4.05-3.89 (m, 1H); 2.54-2.43 (m, 1H); 2.31-2.17 (m, 1H); 1.95-1.79 (m, 1H); 1.78-1.59 (m, 2H); 1.59-1.40 (m, 3H); 1.40-1.24 (m, 1H); 1.06-0.80 (m, 6H)

³¹P NMR (CD₃OD, 202 MHz) 5 (ppm): 51.31

### Step 3: 4-amino-4-[hydroxy(3-methylbutyl)phosphoryl]butanoic acid

The title compound (164 mg, 76%) obtained as a white powder was prepared according to the procedure E for hydrogenolysis from previous product (500 mg, 1.08 mmol, 1.0 eq.) in a mixture EtOH/AcOH (1:1, 18 mL).

Expected purity: >95% (based on LCMS and NMR)

MS (ESI⁺): [(M−H₂O)+H]⁺=220.2; [M+H]⁺=238.2; [(M×2)+H]⁺=475.2;

[(M×3)+H]⁺=712.4

¹H NMR (CD₃OD, 500 MHz) δ (ppm): 3.17-3.04 (m, 1H); 2.62 (t, J=7.5 Hz, 2H); 2.30-2.13 (m, 1H); 2.05-1.83 (m, 1H); 1.74-1.39 (m, 5H); 0.96 (d, J=6.6 Hz, 6H)

³¹P NMR (CD₃OD, 202 MHz) δ (ppm): 33.08

### Example 2

### 4-amino-4-[hydroxy(4-methylpentyl)phosphoryl]butanoic acid

### Step 1: (4-methylpentyl)phosphinic acid

The title compound (740 mg, 43%) was prepared according to the procedure A from diethylchlorophosphite (1.26 mL, 11.5 mmol, 1.0 eq.) in anhydrous Et₂O (6 mL) followed by addition of the freshly prepared Grignard reagent from 1-bromo-4-methylpentane (2.0 g, 12.1 mmol, 1.05 eq.) in anhydrous Et₂O (6 mL).

MS (ESI⁺): [M+H]⁺=151.2; [(M×2)+H]⁺=301.2

¹H NMR (500 MHz, MeOD) δ (ppm): 7.01 (dt, J=536.1, 2 Hz, 1H); 1.78-1.67 (m, 2H); 1.67-1.53 (m, 3H); 1.35-1.27 (m, 2H); 0.91 (d, J=6.6 Hz, 6H)

³¹P NMR (CD₃OD, 202 MHz) δ (ppm): 35.69

### Step 2: [4-(benzyloxy)-1-{[(benzyloxy)carbonyl]amino}-4-oxobutyl](4-methylpentyl) phosphinic acid

The title compound (416 mg, 44%) obtained as a white solid was prepared according to the procedure B for multi-component reaction from previous product (300 mg, 2.0 mmol, 1.0 eq.) and NH₂Cbz (362 mg, 2.4 mmol, 1.2 eq.) in AcOH (5 mL) and AcCl (428 μL) followed by addition of a solution of benzyl 4-oxobutanoate (460.8 mg, 2.4 mmol, 1.2 eq.) in AcOH (5 mL).

MS (ESI⁻): [M−H]⁻=474.2

¹H NMR (500 MHz, MeOD) δ (ppm): 7.39-7.23 (m, 10H); 5.20-5.00 (m, 4H); 3.96 (m, 1H); 2.57-2.43 (m, 2H); 2.27-2.13 (m, 1H); 1.85 (m, 1H); 1.71-1.45 (m, 5H); 1.21 (m, 2H); 0.88 (d, J=6.7 Hz, 6H)

31P NMR (CD₃OD, 202 MHz) δ (ppm): 50.75

### Step 3: 4-amino-4-[hydroxy(4-methylpentyl)phosphoryl]butanoic acid

The title compound (45 mg, 42%) obtained as a beige powder was prepared according to the procedure E for hydrogenolysis from previous product (200 mg, 420 μmmol, 1.0 eq.) in a mixture EtOH/AcOH (1:1, 7 mL).

Expected purity: 95% (based on LCMS and NMR)

MS (ESI⁻): [M−H]⁻=250.2; [(M×2)−H]⁻=501.3; [(M×3)-H]⁻=752.5

MS (ESI⁺): [(M−H20)+H]⁺=234.2; [M+H]⁺=252.2; [(M×2)+H]⁺=503.3; [(M×3)+H]⁺=754.6

¹H NMR (500 MHz, MeOD) δ (ppm): 3.13-3.05 (m, 1H); 2.64-2.56 (m, 2H); 2.27-2.13 (m, 1H); 2.01-1.87 (m, 1H); 1.67-1.51 (m, 5H); 1.29 (q, J=6.9 Hz, 2H); 0.91 (d, J=6.6 Hz, 6H)

³¹P NMR (CD₃OD, 202 MHz) δ (ppm). 32.67

### Example 3

### 4-amino-4-[hydroxy(5-methylhexyl)phosphoryl]butanoic acid

### Step 1: (5-methylhexyl)phosphinic acid

The title compound (797 mg, 46%) was prepared according to the procedure A from diethylchlorophosphite (1.15 mL, 10.54 mmol, 1.0 eq.) in anhydrous Et₂O (6 mL) followed by addition of the freshly prepared Grignard reagent from 1-bromo-5-methylhexane (2.0 g, 11.17 mmol, 1.05 eq.) in anhydrous Et₂O (5 mL).

MS (ESI⁺): [M+H]⁺=165.2; [(M×2)+H]⁺=329.2

¹H NMR (500 MHz, MeOD) δ (ppm): 7.00 (dt, J=533.5, 1.99 Hz, 1H); 1.73 (s, 2H); 1.62-1.51 (m, 3H); 1.43 (dd, J=8.6, 7.5 Hz, 2H); 1.23 (dd, J=8.6, 7.0 Hz, 2H); 0.90 (d, J=6.6 Hz, 6H) ³¹P NMR (CD₃OD, 202 MHz) δ (ppm): 35.5

### Step 2: [4-(benzyloxy)-1-{[(benzyloxy)carbonyl]amino}-4-oxobutyl]5-methylhexyl) phosphinic acid

The title compound (521 mg, 58%) obtained as a white solid was prepared according to the procedure B for multi-component reaction from previous product (300 mg, 1.83 mmol, 1.0 eq.) and NH₂Cbz (331 mg, 2.19 mmol, 1.2 eq.) in AcOH (4 mL) and AcCl (391 μL) followed by addition of a solution of benzyl 4-oxobutanoate (421 mg, 2.19 mmol, 1.2 eq.) in AcOH (3 mL). MS (ESI [M+H]⁺=490.2; [(M×2)+H]⁺=979.7

¹H NMR (500 MHz, MeOD) δ (ppm): 7.32 (dt, J=20.9, 6.2 Hz, 10H); 5.23-4.90 (m, 4H); 4.08-3.84 (m, 1H); 2.72-2.34 (m, 2H); 2.21 (d, J=13.5 Hz, 1H); 1.86 (tt, J=14.0, 7.2 Hz, 1H); 1.72-1.38 (m, 5H); 1.37-1.07 (m, 4H); 0.88 (d, J=6.8 Hz, 6H) 31p NMR (CD₃OD, 202 MHz) δ (ppm): 50.6

### Step 3: 4-amino-4-[hydroxy(5-methylhexyl)phosphoryl]butanoic acid

The title compound (32 mg, 23%) obtained as a beige powder was prepared according to the procedure E for hydrogenolysis from previous product (250 mg, 510 timmol, 1.0 eq.) in a mixture EtOH/AcOH (1:1, 9 mL).

Expected purity: 95% (based on LCMS and NMR)

MS (ESI⁻): [M−H]⁻=264.2; [(M×2)−H]⁻=529.3; [(M×3)−H]⁻=794.6

MS (ESI⁺): [(M−H20)+H]⁺=248.2; [M+H]⁺=266.3; [(M×2)+H]⁺=531.3; [(M×3)+H]⁺=796.6

¹H NMR (500 MHz, MeOD) δ (ppm): 3.13-3.04 (m, 1H); 2.64-2.57 (m, 2H); 2.26-2.14 (m, 1H); 2.00-1.87 (m, 1H); 1.66-1.54 (m, 5H); 1.47-1.36 (m, 2H); 1.27-1.18 (m, 2H); 0.89 (d, J=6.6 Hz, 6H)

³¹P NMR (CD₃OD, 202 MHz) δ (ppm): 32.7

### Example 4

### 4-amino-4-[hydroxy(pentyl)phosphoryl]butanoic acid

### Step 1: pentylphosphinic acid

The title compound (715 mg, 55%) was prepared according to the procedure A from diethylchlorophosphite (1.05 mL, 9.58 mmol, 1.0 eq.) in anhydrous Et₂O (5 mL) followed by addition of pentylmagnesium bromide (2.0 M solution in Et₂O, 5.03 mL, 1.05 eq.).

MS (ESI⁻): [M−H]⁻=135.0

MS (ESI⁺): [M+H]⁺=137.1; [(M×2)+H]⁺=273.1

¹H NMR (500 MHz, MeOD) δ (ppm): 7.01 (dt, J=535.4, 2.0 Hz, 1H); 1.80-1.67 (m, 2H), 1.66-1.53 (m, 2H), 1.49-1.30 (m, 4H), 0.93 (t, J=7.1 Hz, 3H)

³¹P NMR (CD₃OD, 202 MHz) δ (ppm): 35.8

### Step 2: [4-(benzyloxy)-1-{[(benzyloxy)carbonyl]amino}-4-oxobutyl]pentyl)phosphinic acid

The title compound (560 mg, 55%) obtained as a white solid was prepared according to the procedure B for multi-component reaction from previous product (300 mg, 2.2 mmol, 1.0 eq.) and NH₂Cbz (400 mg, 2.64 mmol, 1.2 eq.) in AcOH (9 mL) and AcCl (472 μL) followed by addition of a solution of benzyl 4-oxobutanoate (508 mg, 2.64 mmol, 1.2 eq.) in AcOH (5 mL).

MS (ESI⁻): [M−H]⁻=460.1; [(M×2)−H]⁻=921.5

MS (ESI⁺): [M+H]⁺=462.1; [(M×2)+H]⁺=923.5

¹H NMR (CD₃OD, 500 MHz) δ (ppm): 7.40-7.22 (m, 10H); 5.16-5.02 (m, 4H); 3.96 (m, 1H); 2.57-2.42 (m, 2H); 2.22 (m, 1H); 1.85 (m, 1H); 1.73-1.47 (m, 4H); 1.30 (m, 4H); 0.90 (t, J=5.2, 3.8 Hz, 3H)

³¹P NMR (CD₃OD, 202 MHz) δ (ppm): 50.8

### Step 3: 4-amino-4-[hydroxy(pentyl)phosphoryl]butanoic acid

The title compound (65 mg, 50%) obtained as a beige powder was prepared according to the procedure E for hydrogenolysis from previous product (250 mg, 540 μmol, 1.0 eq.) in a mixture EtOH/AcOH (1:1, 9 mL).

Expected purity: 95% (based on LCMS) and 92% (based on NMR)

MS (ESI⁻): [M−H]⁻=236.2; [(M×2)-H]⁻=473.3; [(M×3)-H]⁻=710.5

MS (ESI⁺): [(M−H₂O)+H]⁺=220.2; [M+H]⁺=238.2; [(M×2)+H]⁺=475.3; [(M×3)+H]⁺=712.5

¹H NMR (500 MHz, MeOD) δ (ppm): 3.12-3.07 (m, 1H), 2.63-2.56 (m, 2H), 2.28-2.14 (m, 1H), 2.00-1.87 (m, 1H), 1.68-1.52 (m, 4H), 1.46-1.31 (m, 4H), 0.99-0.85 (m, 3H)

³¹P NMR (CD₃OD, 202 MHz) δ (ppm). 32.7

### Example 5

### 4-amino-4-[hexyl(hydroxy)phosphoryl]butanoic acid

### Step 1: hexylphosphinic acid

The title compound (1.21 g, 63%) was prepared according to the procedure A from diethylchlorophosphite (1.40 mL, 12.78 mmol, 1.0 eq.) in anhydrous Et₂O (7 mL) followed by addition of hexylmagnesium bromide (2.0 M solution in Et₂O, 6.71 mL, 1.05 eq.).

MS (ESI⁻): [M−H]⁻=149.1

MS (ESI⁺): [M+H]⁺=151.2; [(M×2)+H]⁺=301.2

¹H NMR (500 MHz, MeOD) δ (ppm): 7.01 (dt, J=535.4, 2.0 Hz, 1H); 1.79-1.67 (m, 2H), 1.65-1.52 (m, 2H); 1.50-1.40 (m, 2H); 1.40-1.27 (m, 4H); 0.96-0.87 (m, 3H)

³¹P NMR (CD₃OD, 202 MHz) δ (ppm): 35.8

### Step 2: [4-(benzyloxy)-1-{[(benzyloxy)carbonyl]amino}-4-oxobutyl](hexyl)phosphinic acid

The title compound (572 mg, 60%) obtained as a white solid was prepared according to the procedure B for multi-component reaction from previous product (300 mg, 2.0 mmol, 1.0 eq.) and NH₂Cbz (362 mg, 2.4 mmol, 1.2 eq.) in AcOH (9 mL) and AcCl (428 μL) followed by addition of a solution of benzyl 4-oxobutanoate (460 mg, 2.4 mmol, 1.2 eq.) in AcOH (5 mL).

¹H NMR (500 MHz, MeOD) δ (ppm): 7.41-7.21 (m, 10H); 5.17-5.02 (m, 4H); 4.01-3.91 (m, 1H); 2.57-2.40 (m, 2H); 2.28-2.15 (m, 1H); 1.85 (m, 1H); 1.74-1.46 (m, 4H); 1.38-1.21 (m, 6H); 0.90 (t, J=7.0 Hz, 3H)

³¹P NMR (CD₃OD, 202 MHz) δ (ppm): 50.7

### Step 3: 4-amino-4-[hexyl(hydroxy)phosphoryl]butanoic acid

The title compound (54 mg, 41%) obtained as a beige solid was prepared according to the procedure E for hydrogenolysis from previous product (250 mg, 0.520 mmol, 1.0 eq.) in a mixture EtOH/AcOH (1:1, 9 mL).

Expected purity: 97% (based on LCMS) and 95% (based on NMR)

MS (ESI⁻): [M−H]⁻=250.2; [(M×3)−H]⁺=501.3; [(M×3)-H] =752.6

MS (ESI⁺): [(M−H₂O)+H]⁺=234.2; [M+H]⁺=252.2; [(M×2)+H]⁺=503.3; [(M×3)+H]⁺=754.6

¹H NMR (500 MHz, MeOD) δ (ppm): 3.13-3.04 (m, 1H); 2.64-2.56 (m, 2H); 2.27-2.14 (m, 1H); 2.00-1.86 (m, 1H); 1.69-1.52 (m, 4H); 1.47-1.27 (m, 6H); 0.97-0.86 (m, 3H)

³¹P NMR (CD₃OD, 202 MHz) δ (ppm): 30.7

### Example 6

### 4-amino-4-[hydroxy(4,4,4-trifluorobutyl)phosphoryl]butanoic acid

### Step 1: (4,4,4-Trifluorobutyl)phosphinic acid

The title compound (1 g, 56%) was prepared according to the procedure A from diethylchlorophosphite (1.12 mL, 10.2 mmol, 1.0 eq.) in anhydrous Et₂O (6 mL) followed by addition of the freshly prepared Grignard reagent from 4-bromo-1,1,1-trifluorobutane (2.0 g, 10.0 mmol, 1.05 eq.) in anhydrous Et₂O (5 mL).

MS (ESI⁻): [M−H]⁻=175.1

MS (ESI⁺): [M+H]⁺=177.1; [(M×2)+H]⁺=353.0

¹H NMR (500 MHz, MeOD) δ (ppm): 7.05 (dt, J=537.3, 1.8 Hz, 1H); 2.38-2.24 (m, 2H), 1.90-1.77 (m, 4H)

³¹P NMR (CD₃OD, 202 MHz) δ (ppm): 33.5

### Step 2: [4-(benzyloxy)-1-{[(benzyloxy)carbonyl]amino}-4-oxobutyl](4,4,4-trifluorobut),1) phosphinic acid

The title compound (595 mg, 60%) obtained as a white solid was prepared according to the procedure B for multi-component reaction from previous product (350 mg, 1.99 mmol, 1.0 eq.) and NH₂Cbz (360 mg, 2.39 mmol, 1.2 eq.) in AcOH (9 mL) and AcCl (425 4) followed by addition of a solution of benzyl 4-oxobutanoate (458 mg, 2.38 mmol, 1.2 eq.) in AcOH (5 mL).

MS (ESI³⁰ ): [M+H]⁺=502.1

¹H NMR (500 MHz, MeOD) δ (ppm): 7.43-7.20 (m, 10H); 5.18-5.00 (m, 4H); 4.02-3.91 (m, 1H); 2.60-2.42 (m, 2H); 2.30-2.08 (m, 3H); 1.96-1.64 (m, 5H)

³¹P NMR (CD₃OD, 202 MHz) δ (ppm): 49.1

### Step 3 : 4-amino-4-[hydroxy(4,4,4-trifluorobutyl)phosphoryl]butanoic acid

The title compound (29 mg, 21%) obtained as a beige solid was prepared according to the procedure E for hydrogenolysis from previous product (250 mg, 0.498 mmol, 1.0 eq.) in a mixture EtOH/AcOH (1:1, 9 mL).

Expected purity: 95% (based on LCMS and NMR)

MS (ESI⁻): [M−H]⁻=276.2; [(M×2)-H]⁻=553.2; [(M×3)-H]⁻=830.4

MS (ESI⁺): [(M−H₂O)+H]⁺=260.1; [M+H]⁺=278.2; [(M×2)+H]⁺=555.2; [(M×3)+H]⁺=832.4

¹H NMR (500 MHz, MeOD) δ (ppm): 3.15-3.06 (m, 1H); 2.61 (t, J=7.3 Hz, 2H); 2.36-2.14 (m, 3H); 2.01-1.80 (m, 3H); 1.72-1.58 (m, 2H)

³¹P NMR (CD₃OD, 202 MHz) δ (ppm): 30.9

### Example 7

### 4-amino-4-[(2-cyclohexylethyl)(hydroxy)phosphoryl]butanoic acid

### Step 1: (2-Cyclohexylethyl)phosphinic acid

The title compound (1.2 g, 58%) was prepared according to the procedure A from diethylchlorophosphite (1.29 mL, 11.8 mmol, 1.0 eq.) in anhydrous Et₂O (6 mL) followed by addition of the freshly prepared Grignard reagent from (2-bromoethyl)cyclohexane (2.4 g, 12.6 mmol, 1.05 eq.) in anhydrous Et₂O (6 mL).

MS (ESI⁺): [M+H]⁺=177.2; [(M×2)+H]⁺=353.2

¹H NMR (500 MHz, MeOD) δ (ppm): 7.01 (dt, J=535.8, 1.9 Hz, 1H); 1.82-1.63 (m, 7H); 1.52-1.40 (m, 2H); 1.39-1.13 (m, 4H); 1.02-0.86 (m, 2H)

³¹P NMR (CD₃OD, 202 MHz) δ (ppm): 36.5

### Step 2: [4-(benzyloxy)-1-{[(benzyloxy)carbonyl]amino}-4-oxobutyl](2-cyclohexylethyl) phosphinic acid

The title compound (654 mg, 66%) obtained as a white solid was prepared according to the procedure B for multi-component reaction from previous product (350 mg, 1.99 mmol, 1.0 eq.) and NH₂Cbz (360 mg, 2.39 mmol, 1.2 eq.) in AcOH (9 mL) and AcCl (425 μL) followed by addition of a solution of benzyl 4-oxobutanoate (458 mg, 2.38 mmol, 1.2 eq.) in AcOH (5 mL).

MS (ESI⁻): [M−H]⁻=474.2

MS (ESI⁺): [M+H]⁺=476.2

¹H NMR (500 MHz, MeOD) δ (ppm): 7.41-7.21 (m, 10H); 5.23-4.97 (m, 4H); 3.96 (m, 1H); 2.60-2.42 (m, 2H); 2.32-2.14 (m, 1H); 1.86 (m, 1H); 1.73-1.60 (m, 7H); 1.44 (m, 2H); 1.28-1.10 (m, 4H); 0.85 (p, J=11.6 Hz, 2H)

³¹P NMR (CD₃OD, 202 MHz) δ (ppm): 51.4

### Step 3: 4-amino-4[(2-cyclohexylethyl)(hydroxy)phosphoryl]butanoic acid

The title compound (63 mg, 46%) obtained as a beige solid was prepared according to the procedure E for hydrogenolysis from previous product (250 mg, 0.498 mmol, 1.0 eq.) in a mixture EtOH/AcOH (1:1, 9 mL).

Expected purity: 95% (based on LCMS and NMR)

MS (ESI⁻): [M−H]⁻=276.2; [(M×2)−H]⁻=553.3; [(M×3)−H]⁻=830.6

MS (ESI⁺): [(M−H₂O)+H]⁺=260.2; [M+H]⁺=278.2; [(M×2)+H]⁺=555.3; [(M×3)+H]⁺=832.7

¹H NMR (500 MHz, MeOD) δ (ppm): 3.14-3.04 (m, 1H); 2.64-2.57 (m, 2H); 2.27-2.14 (m, 1H); 2.00-1.86 (m, 1H); 1.82-1.45 (m, 9H); 1.33-1.13 (m, 4H); 1.01-0.86 (m, 2H) ³¹P NMR (CD₃OD, 202 MHz) δ (ppm): 33.1

### Example 8

### 4-amino-4-[(cyclobutylmethyl)(hydroxy)phosphoryl]butanoic acid

### Step 1: (Cyclobutylmethyl)phosphinic acid

The title compound (290 mg, 24%) was prepared according to the procedure A from diethylchlorophosphite (1.26 mL, 11.5 mmol, 1.0 eq.) in anhydrous Et₂O (6 mL) followed by addition of the freshly prepared Grignard reagent from (bromomethyl)cyclobutane (1.4 g, 9.4 mmol, 1.05 eq.) in anhydrous Et₂O (6 mL).

MS (ESI⁺): [M+H]⁺=177

¹H NMR (500 MHz, MeOD) δ (ppm): 6.97 (dt, J=533.5, 2.1 Hz, 1H), 2.76-2.58 (m, 1H); 2.25-2.13 (m, 2H), 1.99-1.76 (m, 6H)

³¹P NMR (CD₃OD, 202 MHz) δ (ppm): 33.1

### Step 2: [4-(benzyloxy)-1-{[(benzyloxy)carbonyl]ainino}-4-oxobutyl](2-cyclobutylethyl) phosphinic acid

The title compound (707 mg, 71%) obtained as a white solid was prepared according to the procedure B for multi-component reaction from previous product (290 mg, 2.16 mmol, 1.0 eq.) and NH₂Cbz (392 mg, 2.59 mmol, 1.2 eq.) in AcOH (5 mL) and AcCl (463 4) followed by addition of a solution of benzyl 4-oxobutanoate (498 mg, 2.59 mmol, 1.2 eq.) in AcOH (4 mL).

MS (ESI⁺): [M+H]⁺=458

¹H NMR (500 MHz, MeOD) δ (ppm): 7.39-7.21 (m, 10H); 5.17-5.01 (m, 4H); 3.89 (s, 1H); 2.64 (m, 1H); 2.48 (m, 2H); 2.26-1.96 (m, 3H); 1.95-1.60 (m, 7H)

³¹P NMR (CD₃OD, 202 MHz) δ (ppm): 49.4

### Step 3: 4-amino-4-[(cyclobutylmethyl)(hydroxy)phosphoryl]butanoic acid

The title compound (45 mg, 35%) obtained as a beige solid was prepared according to the procedure E for hydrogenolysis from previous product (250 mg, 0.544 mmol, 1.0 eq.) in a mixture EtOH/AcOH (1:1, 9 mL).

Expected purity: 97% (based on LCMS) and 95% (based on NMR)

MS (ESI⁻): [M−H]⁻=234.1; [(M×2)−H]⁻=469.2; [(M×3)-H]⁻=704.5

MS (ESI⁺): [(M−H20)+H]⁺=218.2; [M+H]⁺=236.2; [(M×2)+H]⁺=471.2; [(M×3)+H]⁺=706.4

¹H NMR (500 MHz, MeOD) δ (ppm): 3.02-2.96 (m, 1H); 2.75-2.65 (m, 1H); 2.62-2.56 (m, 2H); 2.22-2.14 (m, 3H); 1.97-1.85 (m, 2H); 1.85-1.77 (m, 3H); 1.74 (dd, J=12.9, 7.4 Hz, 2H)

³¹P NMR (CD₃OD, 202 MHz) δ (ppm): 31.1

### Example 9

### 4-amino-4-[(cyclopentylmethyl)(hydroxy)phosphoryl]butanoic acid

### Step 1: (Cyclopentylmethyl)phosphinic acid

The title compound (607 mg, 36%) was prepared according to the procedure A from diethylchlorophosphite (1.26 mL, 11.5 mmol, 1.0 eq.) in anhydrous Et₂O (6 mL) followed by addition of the freshly prepared Grignard reagent from (bromomethyl)cyclopentane (2.0 g, 12.3 mmol, 1.05 eq.) in anhydrous Et₂O (6 mL).

¹H NMR (500 MHz, MeOD) δ (ppm): 7.06 (dt, J=534.5, 2.1 Hz, 1H); 2.20-2.08 (m, 1H), 1.98-1.89 (m, 2H), 1.82 (mm, 2H), 1.73-1.65 (m, 2H), 1.63-1.54 (m, 2H), 1.33-1.21 (m, 2H)

³¹P NMR (CD₃OD, 202 MHz) δ (ppm). 34.4

### Step 2: [4-(benzyloxy)-1-{[(benzyloxy)carbonyl]amino}-4-oxobutyl](cyclopentylmethyl) phosphinic acid

The title compound (541 mg, 56%) obtained as a white solid was prepared according to the procedure B for multi-component reaction from previous product (300 mg, 2.03 mmol, 1.0 eq.) and NH₂Cbz (367 mg, 2.43 mmol, 1.2 eq.) in AcOH (5 mL) and AcCl (433 μL) followed by addition of a solution of benzyl 4-oxobutanoate (467 mg, 2.43 mmol, 1.2 eq.) in AcOH (4 mL).

MS (ESI⁻): [M−H]⁻=472.2

MS (ESI⁺): [M+H]⁺=274.1

¹H NMR (500 MHz, MeOD) δ (ppm): 7.44-7.18 (m, 10H); 5.21-4.97 (m, 4H); 3.93 (m, 1H); 2.57-2.42 (m, 2H); 2.28-2.17 (m, 1H); 2.12 (m, 1H); 1.84 (m, 3H); 1.79-1.69 (m, 2H); 1.67-1.57 (m, 2H); 1.53 (m, 2H); 1.17 (m, 2H)

³¹P NMR (CD₃OD, 202 MHz) δ (ppm): 49.8

### Step 3: 4-amino-4-[(cyclopentylmethyl)(hydroxy)phosphoryl]butanoic acid

The title compound (62 mg, 47%) obtained as a beige solid was prepared according to the procedure E for hydrogenolysis from previous product (250 mg, 0.528 mmol, 1.0 eq.) in a mixture EtOH/AcOH (1:1, 9 mL).

Expected purity: 95% (based on LCMS) and 93% (based on NMR)

MS (ESI⁻): [M−H]⁻=248.2; [(M×2)−H]⁻=497.2; [(M×3)−H]⁻=746.5

MS (ESI⁺): [(M−H₂O)+H]⁺=232.2; [M+H]⁺=250.2; [(M×2)+H]⁺=499.3; [(M×3)+H]⁺=748.5

¹H NMR (500 MHz, MeOD) δ (ppm): 3.10-3.01 (m, 1H); 2.64-2.55 (m, 2H); 2.27-2.12 (m, 2H); 2.02-1.87 (m, 3H); 1.72-1.61 (m, 4H); 1.61-1.51 (m, 2H); 1.31-1.19 (m, 2H)

³¹P NMR (CD₃OD, 202 MHz) δ (ppm): 31.6

### Example 10

### 4-amino-4-[(cyclohexylmethyl)(hydroxy)phosphoryl]butanoic acid

### Step 1: (cyclohexylmethyl)phosphinic acid

The title compound (475 mg, 28%) was prepared according to the procedure A from diethylchlorophosphite (1.15 mL, 10.5 mmol, 1.0 eq.) in anhydrous Et₂O (6 mL) followed by addition of the freshly prepared Grignard reagent from (bromomethyl)cyclohexane (2.0 g, 11.0 mmol, 1.05 eq.) in anhydrous Et₂O (5 mL).

MS (ESI⁺): [M+H]⁺=163.2; [(M×2)+H]⁺=325.2

¹H NMR (500 MHz, MeOD) δ (ppm): 7.01 (dt, J=533.6, 2.2 Hz, 1H); 1.90-1.82 (m, 2H); 1.75-1.62 (m, 6H); 1.34-1.27 (m, 2H); 1.24-1.17 (m, 1H); 1.15-1.04 (m, 2H)

³¹P NMR (CD₃OD, 202 MHz) δ (ppm): 33.7

### Step 2: [4-(benzyloxy)-1-{[(benzyloxy)carbonyl]amino}-4-oxobutyl]cyclohexylmethyl) phosphinic acid

The title compound (501 mg, 55%) obtained as a white solid was prepared according to the procedure B for multi-component reaction from previous product (300 mg, 1.85 mmol, 1.0 eq.) and NH₂Cbz (335 mg, 2.22 mmol, 1.2 eq.) in AcOH (4 mL) and AcCl (396 4) followed by addition of a solution of benzyl 4-oxobutanoate (426 mg, 2.22 mmol, 1.2 eq.) in AcOH (3 mL).

MS (ESI⁺): [M+H]⁺=488.2; [(M×2)+H]⁺=975.6

¹H NMR (500 MHz, MeOD) δ (ppm): 7.40-7.23 (m, 10H); 5.17-5.01 (m, 4H); 3.90 (t, J=9.4 Hz, 1H); 2.56-2.41 (m, 2H); 1.96-1.45 (m, 10H); 1.35-1.20 (m, 3H); 1.06-0.93 (m, 2H)

### Step 3: 4-{[(benzyloxy)carbonyl]amino}-4-[(cyclohexylmethyl)(hydroxy)phosphoryl]butanoic acid

The title compound (205 mg, 100%) obtained as a white solid was prepared according to the procedure C from previous product (250 mg, 0.513 mmol, 1.0 eq.) in a mixture of THF/water (2/1, 5 mL) with presence of LiOH.H₂O (43 mg, 1.03 mmol, 2.0 eq.).

MS (ESI⁻): [M−H]⁻=396.2; [(M×2)−H]⁻=793.4

MS (ESI⁺): [M+H]⁺=398.2; [(M×2)+H]⁺=795.4

¹H NMR (500 MHz, MeOD) δ (ppm): 7.42-7.22 (m, 5H); 5.22-5.03 (m, 2H); 3.97-3.86 (m, 1H); 2.51-2.33 (m, 2H); 2.26-2.13 (m, 1H); 1.92-1.53 (m, 9H); 1.35-1.11 (m, 3H); 1.07-0.93 (m, 2H)

31p NMR (CD₃OD, 202 MHz) δ (ppm): 33.1

### Step 4 : 4-amino-4-[(cyclohexylmethyl)(hydroxy)phosphoryl]butanoic acid

The title compound (27 mg, 20%) obtained as a beige solid was prepared according to the procedure D from previous product (205 mg, 510 μmol, 1.0 eq.) in TFA/anisole (1.5 mL/355 μL).

Estimated purity: 90% (based on NMR)

MS (ESI⁻): [M−H]⁻=262.2; [(M×2)−H]⁻=525.3; [(M×3)-H]⁻=788.6

MS (ESI⁺): [(M−H₂O)+H]⁺=246.2; [M+H]⁺=264.2

¹H NMR (400 MHz, MeOD) δ (ppm): 3.07-2.97 (m, 1H), 2.59 (t, J=7.6 Hz, 2H); 2.28-2.12 (m, 1H); 1.99-1.60 (m, 7H); 1.55-1.46 (m, 2H); 1.40-1.26 (m, 2H); 1.26-1.14 (m, 1H); 1.12-0.99 (m, 2H)

³¹P NMR (CD₃OD, 202 MHz) δ (ppm): 31.8

### Example 11

### Measurement of APA Activity In Vitro

Measurement of APA activity in vitro is based on the protocol of Goldbarg adjusted to the scale of assaying on microplates (Pro Bind™ 3915) (Chauvel et al., 1994). In vitro, in the presence of calcium ions, APA hydrolyses a synthetic substrate α-L-glutamyl-β-naphthylamide (GluβNa) to glutamate and β-naphthylamine ((βNa). A diazotation reaction in acidic medium makes it possible to reveal the β-naphthylamine by formation of a violet-coloured complex: spectrophotometric measurement then makes it possible to know the amount of complex formed and, by reference to a standard curve produced with increasing concentrations of β-naphthylamine, to deduce the enzymatic activity of the sample.

Reagents

The Glu-βNa substrate (Bachem) and the β-naphthylamine (Sigma) are dissolved in 50% DMSO (dimethyl sulphoxide) and 0.1 N HCl respectively, and conserved at −20° C. at a concentration of 10⁻² M. The diazotation reaction is carried out in the presence of sodium nitrite (87 mM), ammonium sulfamate (130 mM) and N-(1-naphthyl)-ethylenediamine dihydrochloride (23 mM in 95% ethanol).

Enzymatic Reaction

The reaction takes place at pH 7.4 in 50 mM tris-HCl buffer, in the presence of calcium (4 mM CaCl₂); recombinant mouse APA is incubated at 37° C. in the presence of the substrate (200 μM Glu-βNa) and in the presence or absence of various concentrations of the inhibitor to be tested, in a final volume of 100 μL. The reaction is stopped by adding 10 μL, of 3N HCl. A standard curve of β-naphthylamine was prepared in parallel by diazotizing increasing concentrations (up to 0.2 mM) of 2-naphthylamine in 0.1 N HCl.

Revelation of the Formed Product

The following are added to each well: 25 μL of sodium nitrite (NaNO₂) (mix, wait 5 minutes at room temperature), 50 μI, of ammonium sulfamate (mix, wait 5 minutes at room temperature), then add 25 μL of N-(1-naphthyl) ethylenediamine dihydrochloride (mix, wait for stabilization of the violet colour for approximately 30 minutes at 37° C.). The absorbance is then measured at 540 nm.

The compound EC33 ((S)-3 amino-4-mercapto-butylsulfonic acid) described in application WO 99/36066 was used as a reference compound.

The results reported in Table 1. show that best compounds (classification a) exhibit the highest APA-inhibiting activity, greater than that of the reference compound by a factor of at least 20.

TABLE1
In vitro inhibition of aminopeptidase A for exemplified inhibitors

| Activity (μM) | Classification |
|---|---|
| IC₅₀ < 0.030 | a |
| 0.030 ≤ IC₅₀ < 0.300 | b |
| 0.300 ≤ IC₅₀ < 10 | c |

| Examples | Results | Examples | Results | Examples | Results |
|---|---|---|---|---|---|
| 8 | a | 1 | b | EC33 | c |
| 9 | a | 2 | b | | |
| 10 | a | 3 | b | | |
| | | 4 | b | | |
| | | 5 | b | | |
| | | 6 | b | | |
| | | 7 | b | | |

1-9. (canceled)

10. A compound having the following formula (I): 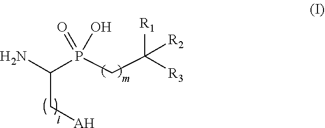
or formula (II): 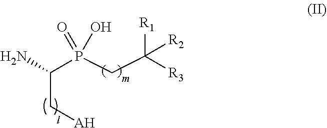
wherein:
AH represents —CO₂H, —SO₃H, —PO₃H₂;
I is 2 or 3;
m is 0,1, 2 or 3;
R₁ represents a halogen atom, an alkyl group, a haloalkyl group, an alkoxy group, a haloalkoxy group, an O-cycloalkyl group, an O-aryl group, an O-arylalkyl group, a heteroalkyl group, an amino group optionally mono or disubstituted by an alkyl group, an haloalkyl a haloalkyl group, a cycloalkyl group, an acyl group, an aryl group or an arylalkyl group;
R₂ and R₃ represent independently a hydrogen atom, a halogen atom, an alkyl group, a haloalkyl group or can form together with the adjacent carbon atom depicted in formula (I) or (II) a cycloalkyl group;
a pharmaceutical salt, solvate, zwitterionic form or prodrug thereof.

11. The compound according to claim 10, wherein the compound corresponds to general formula (I) or formula (II), wherein:
m is 0 or 1; and/or
AH is CO₂H or SO₃H or PO₃H₂; and/or
R₁represents a halogen atom, an alkyl group, a haloalkyl group, an alkoxy group, a haloalkoxy group, an O-cycloalkyl group, an O-aryl group, an O-arylalkyl group, a heteroalkyl group, an haloalkyl a haloalkyl group, a cycloalkyl group, an acyl group, an aryl group or an arylalkyl group.

12. A compound having the following formula (III): 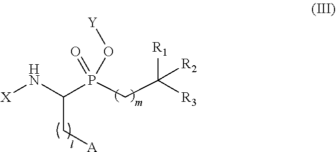
or formula (IV): 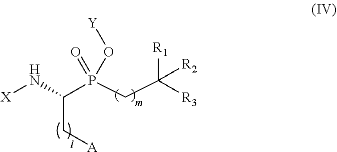
wherein:
I is 2 or 3;
m is 0,1, 2 or 3;
R₁ represents a halogen atom, an alkyl group, a haloalkyl group, an alkoxy group, a haloalkoxy group, an O-cycloalkyl group, an O-aryl group, an O-arylalkyl group, a heteroalkyl group, an amino group optionally mono or disubstituted by an alkyl group, a haloalkyl group, a cycloalkyl group, an acyl group, an aryl group or an arylalkyl group;
R₂ and R₃ represent independently a hydrogen atom, a halogen atom, an alkyl group, a haloalkyl group or can form together with the adjacent carbon atom depicted in formula (I) or (II) a cycloalkyl group;
A represents —SO₃Z —CO₂Z or —P(O)(OZ)₂, with Z selected from the group consisting of a hydrogen atom, an alkyl and arylalkyl group;
X represents a hydrogen atom, —(CO)-alkyl, —(CO)-alkoxy, —(CO)-benzyloxy, 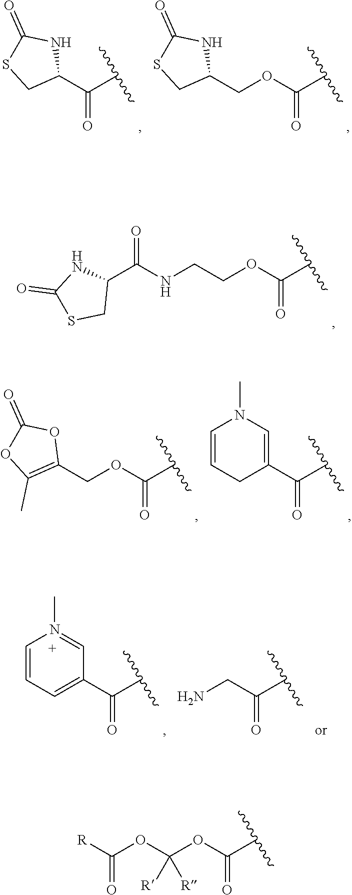
wherein R represents an alkyl group and, R′ and R″ represent independently a hydrogen atom or an alkyl group;
Y represents a hydrogen atom, an alkyl, aryl, arylalkyl or 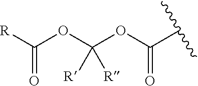
wherein R represents an alkyl group and, R′ and R″ represent independently a hydrogen atom or an alkyl group,
wherein at least one of Z, X and Y is different from a hydrogen atom.

13. The compound according to claim 10, which is selected from the group consisting of:
4-amino-4-[hydroxy(3-methylbutyl)phosphoryl]butanoic acid, 4-amino-4-[hydroxy(4-methylpentyl)phosphoryl]butanoic acid, 4-amino-4-[(2-cyclohexylethyl)(hydroxy)phosphoryl]butanoic acid, 4-amino-4-[hydroxy(pentyl)phosphoryl]butanoic acid, 4-amino-4-[hexyl(hydroxy)phosphoryl]butanoic acid, 4-amino-4-[(cyclobutylmethyl)(hydroxy)phosphoryl]butanoic acid, 4-amino-4-[(cyclopentylmethyl)(hydroxy)phosphoryl]butanoic acid, 4-amino-4-[hydroxy(5-methylhexyl)phosphoryl]butanoic acid, 4-amino-4-[hydroxy(4,4,4-trifluorobutyl)phosphoryl]butanoic acid, 4-amino-4-[(cyclohexylmethyl)(hydroxy)phosphoryl]butanoic acid, and 4-amino-4-[hydroxy({[(propan-2-yl)amino]methyl})phosphoryl]butanoi c acid.

14. A pharmaceutical composition comprising at least one compound according to claim 10 and a pharmaceutically acceptable diluent or carrier.

15-17. (canceled)

18. A method of treatment of a patient suffering from arterial hypertension or directly or indirectly related diseases, comprising the administration to such patient of a therapeutically effective amount of a compound of claim 10.

19. The method of claim 18, wherein the disorders directly or indirectly related to arterial hypertension are selected from the group consisting of heart disease, heart failure, stroke, peripheral and/or cerebral vascular system diseases, brain, eye and/or kidney diseases.

20. The method of claim 18, wherein the disorders are selected from the group consisting of primary and/or secondary arterial hypertension, an ictus, myocardial ischemia, cardiac insufficiency, renal insufficiency, myocardial infarction, a peripheral vascular disease, diabetic protinuria, syndrome X, glaucoma, neurodegenerative diseases and memory disorders.
